(19) United States
(12) Patent Application Publication
(10) Pub. No.: US 2020/0113661 A1
(43) Pub. Date: Apr. 16, 2020

(54) Method For Producing A Dental Prosthesis Part

(71) Applicant: Ivoclar Vivadent AG, Schaan (LI)

(72) Inventor: Christian Ritzberger, Grabs (CH)

(21) Appl. No.: 16/589,179

(22) Filed: Oct. 1, 2019

(30) Foreign Application Priority Data
Oct. 10, 2018 (EP) 18199590.3
Aug. 30, 2019 (EP) 19194640.9

(51) Int. Cl. A61C 13/08; A61C 13/34; A61B 5/00; A61C 13/00; B33Y 50/00; B33Y 80/00; B28B 17/00

(52) U.S. Cl. CPC A61C 13/082; A61C 13/081; A61C 13/34; B28B 17/0081; A61C 13/0019; B33Y 50/00; B33Y 80/00; A61B 5/0088

(57) ABSTRACT

The present invention relates to a method for producing a dental prosthesis part (100), comprising the steps of determining a natural tooth color value with respect to a specified region (101) of the dental prosthesis part (100); and electronically converting the natural tooth color value into a color value of a coloring liquid (103) for coloring the region (101).

### CROSS-REFERENCE TO RELATED APPLICATIONS

This application claims priority to European Patent Application No. 19 194 640.9 filed on Aug. 30, 2019, the disclosure of which is incorporated herein by reference in its entirety. This application also claims priority to European Patent Application No. 18 199 590.3 filed on Oct. 10, 2018, the disclosure of which is incorporated herein by reference in its entirety.

### TECHNICAL FIELD

The present invention relates to a method for producing a dental prosthesis part and to a computer program.

### SUMMARY

Dental prosthesis parts can be designed in terms of color and spatially on computers having corresponding user interfaces (CAD/CAM). In this design process, an attempt is made to determine color values which make the dental prosthesis appear as natural as possible and matched to the patient's remaining teeth. These color values can be determined by means of color detection, i.e. color testing, of adjacent teeth and/or of the remaining vital or non-vital tooth stump prepared by the dentist.

US 20160302896, US 20120052186, US 20120261848, US 20150182317, US 20150086939 and US 20050170315, directed to coloring of dental materials, are hereby incorporated by reference in their entirety.

The colors of a coloring liquid for coloring a partially sintered, porous, dental restoration for the subsequent dental prosthesis are strikingly different from the previously selected natural colors. It is only during the course of the production process, such as for example during sintering, that the coloring liquid for the dental prosthesis part loses its original color and takes on the particular natural color which has been selected or calculated for the dental prosthesis. The coloring liquids are solutions having ions of 3d and/or 4f elements which during sintering are incorporated into the ZrO₂ lattice and effect the coloring procedure. The coloring liquid can comprise one or several food colorants for distinction that are burned out in the process of sintering.

Therefore, the technical object of the present invention is to technically simplify and accelerate the production of a dental prosthesis part.

This object is achieved by the subject matter of the independent claims. Advantageous embodiments are subject-matter of the dependent claims, the description and the figures.

According to a first aspect, this object is achieved by a method for producing a dental prosthesis part, comprising the steps of determining a natural tooth color value with respect to a specified region of the dental prosthesis part; and electronically converting the natural tooth color value into a color value of a coloring liquid for coloring the region.

The method can also be applied to a plurality of regions of the dental prosthesis part with different coloring solutions. The dental prosthesis part is for example a near-net-shape, porous dental restoration which is formed preferably from yttrium-stabilized zirconium oxide. The near-net-shape, porous dental restoration is 15-20% larger than after dense sintering but already has the designated shape of a crown, a bridge, an abutment or an implant. By automatically converting the assigned natural tooth color values into color values of a coloring liquid for the near-net-shape, porous dental restoration, the production of the dental prosthesis part can be simplified and individual coloring can be performed. In the case of 7D-coloring, the coloring is considered in 7 dimensions, three dimensions (3D) for all spatial directions, one dimension (1D) for the a* value, one dimension (1D) for the b* value, one dimension (1D) for the CR value and one dimension (1D) for the L* value.

In a technically advantageous embodiment of the method for producing the dental prosthesis part, the specified region of the dental prosthesis part is automatically colored with the particular coloring liquid which has the converted color value. This provides for example the technical advantage that the production of the dental prosthesis part is accelerated still further. In this technically advantageous embodiment of the method for producing the dental prosthesis part, this can be effected directly in the CAD/CAM machine after milling of the near-net-shape, porous dental restoration, in that the CAD/CAM apparatus is combined with a color application apparatus.

In a further technically advantageous embodiment of the method for producing the dental prosthesis part, the colored dental prosthesis part is optically scanned in order to determine the actual color value of the coloring of the dental prosthesis part with the coloring liquid. This provides for example the technical advantage that the correct coloring of the dental prosthesis part can be verified.

In a further technically advantageous embodiment of the method for producing the dental prosthesis part, the determined actual color value is converted into the allocated natural tooth color value. This also provides for example the technical advantage that the natural appearance of the dental prosthesis part can be pre-calculated on the basis of the actual coloring of the dental prosthesis part.

In a further technically advantageous embodiment of the method for producing the dental prosthesis part, the dental prosthesis part is sintered or densely sintered. This provides for example the technical advantage that the insertable dental prosthesis part is obtained.

In a further technically advantageous embodiment of the method for producing the dental prosthesis part, the color value of the coloring liquid is integrated into a data record which describes the three-dimensional shape of the dental prosthesis part. In addition, the region of the coloring can be integrated into the data record. This also provides for example the technical advantage that the production of the dental prosthesis part is simplified in that a single data record is communicated to a production apparatus.

In a further technically advantageous embodiment of the method for producing the dental prosthesis part, the data record is obtained on the basis of a three-dimensional modelling of the dental prosthesis part. This also provides for example the technical advantage that the data record can be determined in a simple manner.

In a further technically advantageous embodiment of the method for producing the dental prosthesis part, the data record is transmitted to a printing station for coloring the dental prosthesis part. The printing station can be integrated in a CAD/CAM apparatus or exists as a separate apparatus. This also provides the technical advantage that the production of the dental prosthesis part is accelerated further.

In a further technically advantageous embodiment of the method for producing the dental prosthesis part, the color value of the coloring liquid is visually superimposed on data glasses onto the specified region of the dental prosthesis part. By calculating a color scheme and subsequently illustrating it via an augmented-reality-app (AR application) or augmented-reality-data glasses (AR data glasses), the optimum three-dimensional color matching for producing a highly aesthetic dental restoration can be displayed to a person. As a result, each region (point/pixel/surface) can be individually colored. The focus is the 100% individual coloring and color matching to the adjacent teeth with the aid of the AR application (paint-by-numbers) with the color of the adjacent teeth and of the prepared stump having been incorporated beforehand. The aesthetic result is achieved with the aid of the described AR features.

This provides for example the technical advantage that, on the basis of the superimposed false color image, the dental prosthesis part can be manually colored by means of brush infiltration.

In a further technically advantageous embodiment of the method for producing the dental prosthesis part, the color value of the coloring liquid is visually superimposed onto the specified region of the dental prosthesis part on a wearable display apparatus. This also provides for example the technical advantage that, on the basis of the superimposed false color image, the dental prosthesis part can be manually colored by means of brush infiltration.

In a further technically advantageous embodiment of the method for producing the dental prosthesis part, the natural tooth color value is obtained by color-scanning an existing tooth and/or tooth stump. This also provides for example the technical advantage that the natural tooth color values can be determined in a simple manner.

In a further technically advantageous embodiment of the method for producing the dental prosthesis part, a color depth is allocated to the specified region of the displayed dental prosthesis part and the color depth is converted into a number of applications of the coloring liquid. This also provides for example the technical advantage that the desired color depth of the dental prosthesis part can be determined in a simple manner.

According to a second aspect, the object is achieved by a device, comprising means for carrying out the method according to the first aspect. In this way, the same technical advantages can be achieved as by the method according to the first aspect.

According to a third aspect, the object is achieved by a computer program, comprising instructions which, when the computer program is executed by a computer, cause said computer to carry out the method according to the first aspect. In this way, the same technical advantages can also be achieved as by the method according to the first aspect.

Examples of color measuring or detecting devices used herein include, but are not limited to color scanners, colorimeters, spectrophotometers, cameras, and the like.

### BRIEF DESCRIPTION OF THE FIGURES

Exemplified embodiments of the invention are illustrated in the drawings and are described in more detail hereinunder.

In the figures:

FIG. 1 shows a schematic view of a dental prosthesis part; and

FIG. 2 shows a block diagram of the method.

### DETAILED DESCRIPTION

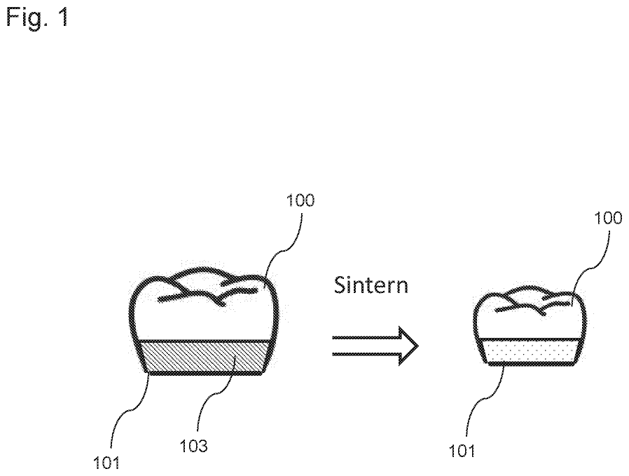

FIG. 1 shows a schematic view of a dental prosthesis part 100.

The dental prosthesis part 100 is for example a crown, a bridge, an abutment or an implant which is given to a patient as a dental prosthesis as part of a dental treatment. In general, the treatment time during the dental treatment is to be kept as short as possible. Therefore, the dental prosthesis part 100 should be available in the shortest time possible.

The dental prosthesis part 100 is produced inter alia in that the milled dental prosthesis part 100 is sintered in a sintering furnace. The near-net-shape, dental restoration is 15-20% larger than after dense sintering but already has the subsequently designated shape. During dense sintering, the near-net-shape, dental restoration assumes the designated size, density, strength and toughness.

Specified regions 101 of the dental prosthesis part 100 are colored with a coloring liquid 103 which changes into the designated natural tooth color only during the course of the sintering process. In contrast to the subsequent natural tooth colors which often differ only in nuances, the different coloring liquids 103 for the dental prosthesis part 100 have colors which in visual terms are highly distinguishable, such as for example quinoline yellow and patent blue or correspond to the color which results from the use of necessary salts of the 3d and 4f elements in the coloring solution. The coloring effect in the sintered dental prosthesis part 100 is generated by ion integration of elements, such as Fe, Er, Tb, Tm, Pr, Co, Cr, Ni, Mn or Bi, into the crystal lattice of the tetragonal and/or cubic zirconium oxide. The partly stabilized or fully stabilized tetragonal and/or cubic zirconium oxide is usually stabilized by Y₂O₃, La₂O₃, CeO₂, MgO or CaO or their mixtures. In the coloring liquids 103 salts, such as chlorides, nitrates, of the 3d- or 4f-elements are dissolved. Distinction of coloring liquids in terms of color can be achieved by using suitable food colorants.

During the production of the dental prosthesis part, it is time-consuming to determine and apply the coloring solutions 103 which are to be used. However, the production time for the dental prosthesis part 100 should be shortened to such an extent that a dental treatment involving the dental prosthesis can be performed for example in half a day.

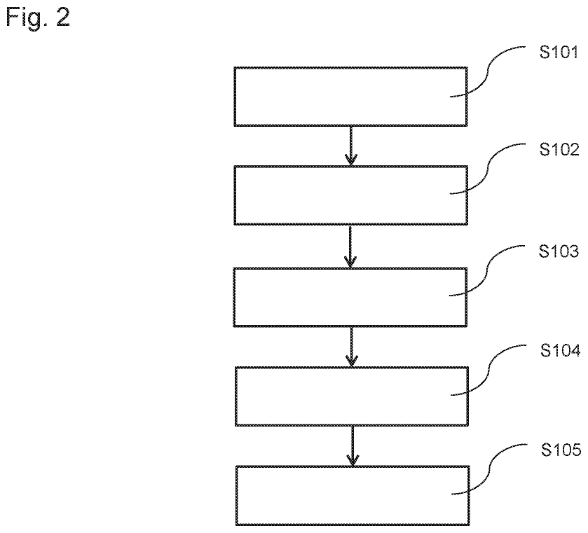

FIG. 2 shows a block diagram of the method for producing the dental prosthesis part 100 in an accelerated manner.

Firstly, in step S101 a specific blank consisting of zirconium oxide is provided in dependence upon the respective dental indication, such as for example for use as a front or lateral tooth, crown or bridge, with a holder for milling purposes. This blank is also defined as a white blank because it is not pre-colored. The blank is debindered and already pre-sintered.

For the different dental indications, only three different zirconium oxide blanks are used which differ in particular in their Y₂O₃ proportion. The Y₂O₃ proportion has an influence upon the translucency and strength of the final dental prosthesis part 100. In addition to Y₂O₃, other oxides can be used for stabilization purposes, for example oxides of Ce, Mg, Ca and La as well as mixtures thereof can be used. A first type of zirconium oxide blank comprises 2.5-3.5 mol. % of yttrium-stabilized zirconium oxide. A second type of zirconium oxide blank comprises 3.5-4.5 mol. % of yttrium-stabilized zirconium oxide. A third type of zirconium oxide blank comprises 4.5-5.5 mol. % of yttrium-stabilized zirconium oxide. Depending upon the dental indication, a selection is made from the three different zirconium oxide blanks. This selection can be made quickly and easily by a dentist or by suitable software.

In step S102, the shape of the dental prosthesis part 100 is defined. For this purpose, the tooth region which is to be worked on is prepared with one or a plurality of teeth. After the preparation is concluded, a digital scan is performed by means of an intraoral camera.

Using the digital data, the restoration, i.e. the dental prosthesis part 100, is modelled by means of a CAD program. Then, the modelled shape of the dental prosthesis part 100 is milled from the previously selected zirconium oxide blank in a CAD/CAM process.

The dental prosthesis part 100 thus obtained is formed by a near-net-shape, partially sintered, porous dental restoration. The near-net-shape, dental restoration is 15-20% larger than after subsequent dense sintering but already has the designated shape of the subsequent crown, the bridge, the abutment or the implant.

In step S103, the natural tooth color values are determined for specified regions 101 of the dental prosthesis part 100. The natural tooth color values for the dental prosthesis part 100 can be determined by a color testing of the adjacent teeth and a remaining tooth stump on the basis of an optical measuring method using a camera. The tooth stump can be vital and/or non-vital. Using the data obtained thereby, software calculates a 3D model for the individual natural coloring and shape of the dental restoration so that, in terms of color, said restoration is incorporated seamlessly into the remaining teeth.

In step S104, the determined natural tooth color values are converted electronically into color values of coloring liquids 103 for coloring the dental prosthesis part 100 prior to sintering. The conversion can be effected on the basis of a pre-stored table in which the respective color values of the coloring liquids 103 are allocated to the respective natural tooth color values. The color values of the coloring liquids 103 can be provided in the form of an image which provides the dentist with simple guidance as to which regions 101 of the dental prosthesis part 100 are to be colored, and how, by means of the coloring liquids 103, i.e. infiltration colors. For this purpose, a freely rotatable 3D model can be shown on a display screen, said model displaying the colors of the coloring liquids 103 for the respective regions 101 on a display screen. However, the 3D model can be shown by means of AR-software (AR—Augmented Reality) on a wearable apparatus including a camera, such as for example a smartphone or tablet, or on data glasses, and can be visually superimposed upon the dental prosthesis part 100. As a result, it is also easily possible to recognize which regions 101 of the dental prosthesis part 100 are to be colored, and how, by means of the coloring liquids 103.

The data glasses are an electronic apparatus which are worn as glasses and can be used to optically display the color values for the respective regions 101 to a user in addition to natural visual perception of the dental prosthesis part 100. By means of the display device, the data glasses can superimpose additional information on an image which is perceived by the wearer's eye. The display device comprises for example a display screen close to the eyes or a projector for directly projecting on the retina. In a corresponding manner, this can also be performed using a wearable apparatus.

The 3D model is shown in such a way that color regions 101 of the displayed 3D model correspond to the color of the coloring solutions 103, with which the dental prosthesis part 100 has to be colored in order to subsequently obtain the desired natural appearance. As a result, the dental prosthesis part 100 can be easily colored accordingly and the time for producing the dental prosthesis part 100 can be reduced.

The specified regions 101 of the dental prosthesis part 100 can be colored automatically with the respective coloring liquids 103 which have the converted color values. This can be effected by using a printing apparatus, with which the coloring liquids 103 are applied to the dental prosthesis part. The regions 101 can also be colored manually by means of brush infiltration on the basis of the displayed image. Alternatively, coloring liquids 103 can be sprayed up by means of a suitable spraying device.

Then, the colored dental prosthesis part 100 can be scanned optically once again by means of a measuring method in order to determine the actual color values of the coloring of the dental prosthesis part 100 with the coloring liquids 103 and to compare them with the designated color values of the coloring liquids 103. It is possible to verify that each of the specified regions 101 is colored with the designated coloring liquid. As a result, correct coloring can be verified. The determined actual color values of the coloring of the dental prosthesis part 100 with the coloring liquids 103 can be converted back to the respectively allocated natural tooth color values. In this manner, a subsequent appearance can be determined and displayed on the basis of the actually colored dental prosthesis part 100. The measuring methods can be performed by a camera or a suitable RGB sensor. Thereby it is possible to determine the coloring of the dental prosthesis part 100. The a*-value, the b*-value, the CR-value and the L*-value can be determined. It is conceivable that only a single value is determined from the preceding values and is back calculated into each allocated natural tooth color value. Conversion can be performed by a computer program and displayed on a screen.

The converted color values of the coloring liquid 103 and the associated regions 101 can be integrated into a data record which describes the three-dimensional shape of the dental prosthesis part 100. This can be obtained on the basis of a three-dimensional scan of a mouth position or a digital modelling. This data record can then be transmitted to a printing station or a printing apparatus for automatically coloring the dental prosthesis part 100.

A color depth can be allocated to each specified region 101 of the dental prosthesis part 100 either automatically or by means of a user interface. This established color depth can be converted automatically into a number of applications of the coloring liquid 103. Then, the coloring liquid 103 can be applied correspondingly often, for example by means of the printing apparatus.

In step S105, the dental prosthesis part 100 is dried and sintered in a furnace. The drying can be effected in a furnace or with an infrared source. Since infiltration takes place only superficially, the drying can be effected in a time range of 10 s to 10 min. The sintering process gives the dental prosthesis part 100 its final shape and the coloring liquids 103 produce the designated natural tooth color values in the densely sintered state. Then, surface polishing or glaze baking can be effected for the purpose of surface finishing.

The method renders it possible to provide patients with a dental restoration consisting of zirconium oxide in a short amount of time. Said restoration has a coloring which corresponds precisely to the intended coloring.

Determining and converting the natural tooth color values into color values of coloring liquids results in a considerable reduction in the time required for producing the dental prosthesis part 100 in addition to the other measures. As a result, the time required for a dental treatment in which the dental prosthesis part 100 is used can be reduced considerably.

All features explained and illustrated in conjunction with individual embodiments of the invention can be provided in different combinations in the subject matter in accordance with the invention in order to achieve the advantageous effects thereof at the same time.

All the method steps can be implemented by devices which are suitable for carrying out the respective method step. All functions which are carried out by apparatus features can be a method step of a method.

The scope of protection of the present invention is set by the claims and is not limited by the features explained in the description or shown in the figures.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. Other programming paradigms can be used, e.g., functional programming, logical programming, or other programming. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation.

Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

In some embodiments, the system may include a camera, a processor, an electronic data storage unit, and a display. The camera can be a standard camera, an infrared dot-projection detector, flood illuminator camera, structured-light three-dimensional scanner, standard infrared detector, ultrasonic imaging device, Doppler detector, or any other suitable visualization system capable of capturing information related to a patient's dentition. The processor can be a single processor having one or more cores, or a plurality of processors connected by a bus, network, or other data link. The electronic data storage unit can be any form of non-transitory computer-readable storage medium suitable for storing the data produced by the system. The display can be any display suitable for displaying a digital color or grayscale image.

In some embodiments, the camera, processor, electronic data storage unit, and digital display are components of a single device. The single device may be a smartphone, tablet, laptop computer, personal digital assistant, or other computing device.

In some embodiments, the processor is in communication over a network, which could be wired or wireless, with an external processor used for performing one or more calculation steps and/or a network-attached electronic data storage unit.

In some embodiments, the present disclosure makes use of cloud computing to perform one or more calculations steps remotely and/or remote storage to enable the storage of data remotely for collaborative or remote analysis.

In some embodiments, the system comprises a plurality of graphical user interfaces to permit multiple users to view or analyze the same data.

In some embodiments, the system operates by capturing information related to a patient's dentition using a camera, creating a model of the patient's dentition on a processor, fitting a model of a proposed post-alteration dentition to the patient's dentition on the processor, coloring the model of the proposed post-alteration dentition to match an expected real post-alteration coloration, and displaying the fitted model of the proposed post-alteration dentition in place of the patient's actual dentition on a display which otherwise shows the patient's actual facial features. The information related to a patient's dentition, the model of the patient's dentition, and the model of the proposed post-alteration dentition may be stored on an electronic data storage unit.

In some embodiments, the operations are performed in real-time.

In some embodiments, a user interface is configured such that a user may view the “before” dentition image and the “after” dentition image simultaneously either side-by-side or with a full or partial overlay.

Where used herein, the term “non-transitory” is a limitation on the computer-readable storage medium itself—that is, it is tangible and not a signal—as opposed to a limitation on the persistence of data storage. A non-transitory computer-readable storage medium does not necessarily store information permanently. Random access memory (which may be volatile, non-volatile, dynamic, static, etc.), read-only memory, flash memory, memory caches, or any other tangible, computer-readable storage medium, whether synchronous or asynchronous, embodies it.

Although the invention is illustrated above, partly with reference to some preferred embodiments, it must be understood that numerous modifications and combinations of different features of the embodiments can be made. All of these modifications lie within the scope of the appended claims.

1. Method for producing a dental prosthesis part (100), comprising the steps of:
determining (S103) a natural tooth color value with respect to a specified region (101) of the dental prosthesis part (100); and
electronically converting (S104) the natural tooth color value into a color value of a coloring liquid (103) for coloring the region (101).

2. Method as claimed in claim 1,
wherein the specified region (101) of the dental prosthesis part (100) is colored automatically with the coloring liquid (103) which has the converted color value.

3. Method as claimed in claim 2,
wherein the colored dental prosthesis part (100) is optically scanned in order to determine the actual color value of the coloring of the dental prosthesis part (100) with the coloring liquid (103).

4. Method as claimed in claim 3,
wherein the determined actual color value is converted into the allocated natural tooth color value.

5. Method as claimed in claim 1,
wherein the dental prosthesis part (100) is sintered.

6. Method as claimed in claim 1,
wherein the color value of the coloring liquid (103) is integrated into a data record which describes the three-dimensional shape of the dental prosthesis part (100).

7. Method as claimed in claim 6,
wherein the data record is obtained on the basis of a three-dimensional modelling of the dental prosthesis part (100).

8. Method as claimed in claim 7,
wherein the data record is transmitted to a printing station for coloring the dental prosthesis part (100).

9. Method as claimed in claim 1,
wherein the color value of the coloring liquid (103) is visually superimposed on data glasses onto the specified region of the dental prosthesis part (100).

10. Method as claimed in claim 1,
wherein the color value of the coloring liquid (103) is visually superimposed on a wearable display apparatus onto the specified region of the dental prosthesis part (100).

11. Method as claimed in claim 1,
wherein the natural tooth color value is obtained by color-scanning an existing tooth and/or tooth stump.

12. Method as claimed in claim 1,
wherein a color depth is allocated to the specified region (101) of the displayed tooth prosthesis part (100) and the color depth is converted into a number of applications of the coloring liquid (103).

13. Method as claimed in claim 1,
wherein the natural tooth color value is determined using a color detecting device;
wherein a processor is in electronic communication with the color detecting device; and
wherein the processor electronically converts (S104) the natural tooth color value into a color value of a coloring liquid (103).

14. A device comprising means for carrying out the method as claimed in claim 1 comprising
a color detecting device for detecting a tooth color; and
a processor in communication with the color detecting device for electronically converting the tooth color to a color value of a coloring liquid.

15. Computer program, comprising instructions which, when the computer program is executed by a computer, cause the computer to carry out the method as claimed in claim 1.
